(19) United States
(12) Patent Application Publication
(10) Pub. No.: US 2023/0172980 A1
(43) Pub. Date: Jun. 8, 2023

(54) Chimeric Antigen Receptor, Construction Method Therefor and Application Thereof

(71) Applicant: Fundamenta Therapeutics Co., Ltd., Wujiang District, Suzhou, Jiangsu (CN)

(72) Inventors: Jun Li, Wujiang District, Suzhou, Jiangsu (CN); Pengchao Zhang, Wujiang District, Suzhou, Jiangsu (CN); Zhao Xu, Wujiang District, Suzhou, Jiangsu (CN); Ying Chen, Wujiang District, Suzhou, Jiangsu (CN); Linmao Zhong, Wujiang District, Suzhou, Jiangsu (CN); Yuchen Jiang, Wujiang District, Suzhou, Jiangsu (CN); Ling He, Wujiang District, Suzhou, Jiangsu (CN)

(73) Assignee: Fundamenta Therapeutics Co., Ltd., Wujiang District, Suzhou, Jiangsu (CN)

(21) Appl. No.: 17/635,170

(22) PCT Filed: Aug. 13, 2020
(86) PCT No.: PCT/CN2020/108850

§ 371 Date: Jul. 26, 2022

(30) Foreign Application Priority Data
Aug. 14, 2019 (CN) 201910748321.2

(51) Int. Cl. A61K 35/17; C12N 15/85; C07K 16/28; A61P 35/02

(52) U.S. Cl. CPC A61K 35/17; C12N 15/85; C07K 16/2815; C07K 16/2818; A61P 35/02

(57) ABSTRACT

Provided are a chimeric antigen receptor, a construction method therefor and an application thereof. The chimeric antigen receptor consists of an antigen binding domain, an extracellular hinge region, a transmembrane domain, a co-stimulatory domain and a CD3z signaling domain. Further provided is a CAR-T cell which comprises two chimeric antigen receptors containing different antigen binding domains, and which is bispecific and exhibits increased cell killing efficiency and a better tumor inhibitory effect in vivo.

### SEQUENCE LISTING SUBMISSION VIA EFS-WEB

A computer readable text file, entitled “SequenceListing.txt,” created on Mar. 29, 2022 with a file size of 35,753 bytes contains the sequence listing for this application and is hereby incorporated by reference in its entirety.

### PRIORITY AND RELATED APPLICATION

The present application claims the priority of Chinese patent application 201910748321.2 entitled “Chimeric Antigen Receptor, Construction Method Therefor and Application Thereof” filed on Aug. 14, 2019, and the entire contents of this application, including appendix, are incorporated into herein by reference.

### TECHNICAL FIELD

The present disclosure belongs to the field of biotechnology, and specifically relate to a chimeric antigen receptor as well as the construction method and use thereof.

### BACKGROUND

With the development of tumor treatment, chimeric antigen receptor-T (CAR-T) cellular immunotherapy has gradually become a treatment method that has attracted much attention. CAR expressed by CAR-T cell generally comprises an extracellular antigen binding domain, a transmembrane domain, a co-stimulator domain and an intracellular signaling domain. In general, CAR-T cells are obtained by the transduction of CAR gene into T cells derived from a patient and the cell expansion, and are finally reinfused into the patient. CAR-T cells are capable of effectively recognizing tumor antigens and eliciting specific antitumor immune response without being restricted by the major histocompatibility complex (MHC). Currently, the US FDA has approved two autologous CAR-T cell products, namely Kymriah from Novartis and YesCAR-Ta from Kite, which are used for the treatment of acute lymphoblastic leukemia (ALL) and refractory/relapsed non-Hodgkin lymphoma, respectively. A large number of clinical trials have proved that CAR-T has great antitumor potential as a personalized living-cell drug (Maude et al., 2018; Park et al., 2018; Schuster et al., 2017).

Although a variety of tumor antigens are being applied in clinical research, CD19 is still the most widely used target in CAR-T research. Although CAR-T therapy has achieved unprecedented efficacy in the treatment of hematological tumors, there are still some patients who do not respond to CAR-19-T, or there remains great problem in terms of the persistence of CAR-19-T even if the initial treatment produces certain efficacy. Some studies have indicated that the limited efficacy of CAR-T is partly due to the loss or downregulation of tumor cell surface antigens (Grupp et al., 2013; Ruella et al., 2016). Neelapu reported the Phase 2 clinical results of their CAR-19-T product in non-Hodgkin lymphoma, 108 patients were followed up to at least one year after CAR-T therapy. Among them, 42% of the patients responded well, however, there were still some patients relapsed, and among these, the immune escape caused by CD19-negative relapse was the main reason (Neelapu et al., 2017). In the treatment of acute lymphoma, the overall response rate of CAR-19-T was 81%, and the majority of relapsed patients (15/22) showed CD19-negative escape (Kantarjian et al., 2016). Therefore, target antigen escape is currently one of the important bottlenecks in CAR-T therapy.

The main approach to address antigen loss during CAR-T therapy is targeting multiple antigens, thereby addressing the negative escape of a single tumor antigen. In a preclinical study of CAR-T in glioma, the researchers first attempted the combination therapy of multiple-targeting CAR-T cells. As compared with single-targeting CAR-T cells, dual-targeting CAR-T cells targeting HER2 and IL-23Rα2 were capable of significantly preventing antigen escape and achieving better tumor inhibitory effect (Hegde et al., 2013). In a preclinical study of dual-targeting CAR-T in B-cell malignancy, Zah developed a CD19-CD20 tandem dual-targeting CAR-T cell, which was capable of significantly inhibiting the spontaneous escape of CD19-negative tumor cells in immunodeficient mice (Zah et al., 2016). CARs in a tandem dual-targeting CAR-T cell share one signal transducer while CARs in a combinational dual-targeting CAR-T cell respectively depend on their own signal transducer (please refer to FIG. 1). Therefore, as compared with the tandem dual-targeting CAR-T cell, the signals of the combinational CAR-T cell are independent and will not interfere with each other, thereby capable of exerting the therapeutic effect of the CAR-T cell in the most efficient manner. Plue conducted related research on a combinational dual-targeting CAR19-CAR22 (WO2016/102965A1), and found that the dual-targeting CAR19-CAR22 combination of 4-1BBzeta/OX40zeta and CD28zeta had the optimal in-vitro tumoricidal effect, indicating that different combinations of intracellular stimulators in the combinational dual-targeting CAR-T cell would result in different tumor inhibitory effects in vivo and in vitro. Therefore, as for the combinational dual-targeting CAR-T cell, it is extremely crucial to find the most suitable combination among a variety of different extracellular antigen binding domains, transmembrane domains and co-stimulator domains.

The combinational bispecific CAR-T cell provided in the present disclosure adopts two independent chimeric antigen receptors, and has the advantages of having independent signal and having no mutual influence as compared with a tandem bispecific CAR-T cell. In the invention patent WO2016/102965A1, researches on tumor targets, i.e., CD19 and CD22 were conducted. The in-vitro tumoricidal activities of four combinations of intracellular stimulators (including 41BBz-41BBz, OX40z-OX40z, 41BBz-28z and OX40z-28z) were compared in the combinational dual-targeting CAR19-CAR22 in this patent. However, in this patent, the screened data is a single one with few combinations, and there is no relevant in-vivo data. In the present disclosure, researches on tumor targets, i.e., CD19, CD20 and CD22 are conducted, and the comparison and confirmation of the in-vivo and in-vitro tumoricidal activities of the combinational dual-targeting CAR19-CAR20 and the combinational dual-targeting CAR19-CAR22 with multiple combinations are conducted in a more systematic way.

### SUMMARY

### Problems to be Solved by the Disclosure

In view of the problems existing in the prior art, the present application provides a series of specific chimeric antigen receptors as well as the construction method and use thereof, in particular, provides a bispecific CAR-T cell that comprises two chimeric antigen receptors comprising different antigen binding domains.

### Means for Solving the Problems

In view of the above-mentioned problems existing in the prior art, the present inventors have conducted intensive studies and repeated experiments, so as to conduct systematical optimization and comparison among the extracellular hinge regions, the transmembrane domains and the co-stimulator domains that respectively have different structures in CAR20 and CAR22 of the combinational bispecific chimeric antigen receptor CAR19-CAR20 and the combinational bispecific chimeric antigen receptor CAR19-CAR22, thereby completing the present disclosure. That is, the present disclosure is described as follows.

In the first aspect of the present disclosure, first, provided is a chimeric antigen receptor composed of an antigen binding domain, an extracellular hinge region, a transmembrane domain, a co-stimulatory domain and a CD3z signaling domain.

For those skilled in the art, said “co-stimulatory domain” (CSD) may also be referred to as a co-stimulator or a co-stimulator domain.

In specific embodiments of the present disclosure, the extracellular hinge region is any one selected from the group consisting of CD8 extracellular hinge region (CD8hinge), CD28 extracellular hinge region (CD28hinge), ICOS extracellular hinge region (ICOShinge) and IgG4mt10+N297A extracellular hinge region (IgG4mt10+N297Ahinge);

the transmembrane domain is any one selected from the group consisting of CD8 transmembrane domain (CD8TM), CD28 transmembrane domain (CD28TM) and ICOS transmembrane domain (ICOSTM); and

the co-stimulatory domain is any one selected from the group consisting of 4-1BB co-stimulatory domain (4-1BBCSD), CD28 co-stimulatory domain (CD28CSD), ICOS co-stimulatory domain (ICOSCSD) and OX40 co-stimulatory domain (OX40CSD).

In specific embodiments of the present disclosure, the structures comprising the extracellular hinge region, the transmembrane domain and the co-stimulatory domain are respectively as follows: CD8hinge-CD8TM-4-1BBCSD, CD28hinge-CD28TM-CD28CSD, ICOShinge-ICOSTM-ICOSCSD, CD28hinge-CD28TM-OX40CSD, IgG4mt10+N297Ahinge-CD8TM-4-1BBCSD, IgG4mt10+N297Ahinge-CD28 TM-CD28CSD, or IgG4mt10+N297Ahinge-ICOSTM-ICOSCSD.

In the above formulae, “-” is independently a linker peptide or a peptide linkage; “hinge” denotes a hinge region; TM denotes a transmembrane domain; and CSD denotes a co-stimulatory domain.

Among them, the amino acid sequence of the IgG4mt10+N297Ahinge-CD8TM-4-1BBCSD is SEQ ID NO: 36, and the nucleotide sequence encoding the amino acid sequence is SEQ ID NO: 33;

the amino acid sequence of the IgG4mt10+N297Ahinge-CD28TM-CD28CSD is SEQ ID NO: 37, and the nucleotide sequence encoding the amino acid sequence is SEQ ID NO: 34; and

the amino acid sequence of the IgG4mt10+N297Ahinge-ICOSTM-ICOSCSD is SEQ ID NO: 38, and the nucleotide sequence encoding the amino acid sequence is SEQ ID NO: 35.

The antigen binding domain comprised in the chimeric antigen receptor as described above is a single-chain antibody (scFv) or a single domain antibody (sdAb).

Among these, the scFv is formed by linking the heavy chain variable region and the light chain variable region of an antibody via a short-chain peptide (linker) of 15 to 20 amino acids. The single domain antibody is also referred to as a nanobody or a heavy chain antibody (hcAb), and its volume is approximately 1/10 of a traditional antibody. Unlike a traditional antibody, a single domain antibody is merely composed of a heavy chain, its antigen binding domain is merely a single domain connected to the Fc region via a hinge region, and this antigen binding domain still has the function to bind antigen after being isolated from the antibody.

In specific embodiments of the present disclosure, the antigen binding domain recognizes CD20 or recognizes CD22.

Further, the antigen binding domain is Leu16, wherein said Leu16 is a humanized scFv recognizing CD20 and the amino acid sequence of said Leu16 is as set forth in SEQ ID NO: 3.

Alternatively, the antigen binding domain is M971, wherein said M971 is an scFv recognizing CD22 and the amino acid sequence of said M971 is as set forth in SEQ ID NO: 7.

In the second aspect of the present disclosure, provided is a chimeric antigen receptor T cell, i.e., CAR-T cell, the CAR-T cell is capable of expressing any one of the specific chimeric antigen receptors of the first aspect of the present disclosure, wherein the CAR-T cell expresses two independent chimeric antigen receptors.

In one embodiment of the present disclosure, the two independent chimeric antigen receptors are respectively CAR19 and CAR20, wherein said CAR19 recognizes CD19 and said CAR20 recognizes CD20.

In another embodiment of the present disclosure, the two independent chimeric antigen receptors are respectively CAR19 and CAR22, wherein said CAR19 recognizes CD19 and said CAR22 recognizes CD22.

In the third aspect of the present disclosure, provided is a nucleic acid molecule encoding any one of the specific chimeric antigen receptors of the first aspect of the present disclosure.

In the fourth aspect of the present disclosure, provided is a vector comprising the nucleic acid molecule of the third aspect of the present disclosure.

In the fifth aspect of the present disclosure, provided is a host cell, the host cell comprises the vector of the fourth aspect of the present disclosure or the chromosome of the host cell is integrated with the nucleic acid molecule of the third aspect of the present disclosure.

In the sixth aspect of the present disclosure, provided is a pharmaceutical composition comprising a pharmaceutically acceptable vector and any one of the specific chimeric antigen receptors of the first aspect of the present disclosure.

In the seventh aspect of the present disclosure, provided is the use of any one of the specific chimeric antigen receptors of the first aspect of the present disclosure, the nucleic acid molecule of the third aspect of the present disclosure, the vector of the fourth aspect of the present disclosure or the host cell of the fifth aspect of the present disclosure in the preparation of an antitumor drug or an antitumor preparation.

Among these, the tumor is a hematological tumor, preferably, the hematological tumor is B-cell malignancy, acute lymphocytic leukemia, chronic lymphocytic leukemia, lymphoma, mastocytoma or follicular lymphoma.

In the eighth aspect of the present disclosure, provided is a method for preparing a CAR-T cell, wherein the CAR-T cell expresses the specific chimeric antigen receptor of the present disclosure, and the method comprises the following step:

introducing the nucleic acid molecule of the third aspect of the present disclosure or the vector of the fourth aspect of the present disclosure into a T cell, so as to obtain the CAR-T cell.

### Advantageous Effects of the Disclosure

1. The lentiviral vector comprising the nucleic acid molecule encoding a combinational bispecific chimeric antigen receptor CAR19-CAR20 provided in the present disclosure is capable of infecting human T lymphocytes in vitro, and 7 kinds of CAR19-CAR20-T cells comprising bispecific chimeric antigen receptors with different structures have relatively high killing efficiency for both CD19⁺K562-luc-GFP target cells and CD20⁺K562-luc-GFP target cells. Among these, CAR19-CAR20-T cells comprising PCTL152 and CAR19-CAR20-T cells comprising PCTL153 still have relatively high killing efficiency for target cells in a case where the effector-to-target ratio is relatively low.

2. All the combinational bispecific chimeric antigen receptor CAR19-CAR20-T cells provided in the present disclosure are capable of maintaining a relatively high proportion of stem cell-like central memory T cells (TSCMs). Among these, CAR19-CAR20-T cells comprising PCTL152 and CAR19-CAR20-T cells comprising PCTL153 are capable of better expressing CAR19⁺CAR20⁺ dual-positive population.

3. All the combinational bispecific chimeric antigen receptor CAR19-CAR20-T cells provided in the present disclosure have good in-vivo tumor inhibitory effect. In particular, as compared with CAR19-CAR20-T cells comprising PCTL152, CAR19-CAR20-T cells comprising PCTL153 are capable of improving the survival rate of tumor-bearing mice more significantly; as compared with traditional single-targeting CAR19-T cells, single-targeting CAR20-T cells and tandem CAR20-19-T cells, the combinational dual-targeting CAR19-CAR20-T cells comprising PCTL153 are capable of improving the survival rate of tumor-bearing mice significantly.

4. All the combinational bispecific chimeric antigen receptor CAR19-CAR22-T cells provided in the present disclosure have a killing efficiency of more than 90% for CD19⁺K562-luc-GFP target cells in a case where the effector-to-target ratio is 10:1. In addition, said bispecific chimeric antigen receptor CAR19-CAR22-T cells have killing effects on CD22⁺K562-luc-GFP target cells under a series of different effector-to-target ratios, and the killing effects are apparently effector-to-target ratio-dependent.

In order to enable the above-mentioned and other purposes, features and advantages of the present disclosure to be more apparent and easier to understand, preferred examples are exemplified below and described in detail as follows in conjunction with the accompanying drawings.

### BRIEF DESCRIPTION OF THE DRAWINGS

FIG. 1 shows the schematic diagram of the structures of a tandem bispecific CAR-T and a combinational bispecific CAR-T.

FIG. 2 shows the schematic diagram of the structures of 7 kinds of combinational bispecific chimeric antigen receptor CAR19-CAR20s.

FIG. 3 shows the killing efficiency of 7 kinds of CAR19-CAR20-T cells comprising bispecific chimeric antigen receptors with different structures for CD19⁺K562-luc-GFP target cells.

FIG. 4 shows the killing efficiency of 7 kinds of CAR19-CAR20-T cells comprising bispecific chimeric antigen receptors with different structures for CD20⁺K562-luc-GFP target cells.

FIG. 5 shows the T cell phenotypes of 7 kinds of CAR19-CAR20-T cells comprising bispecific chimeric antigen receptors with different structures.

FIG. 6 shows the results illustrating CAR19⁺CAR20⁺ dual-positive population expressed by CAR19-CAR20-T cells comprising PCTL152 and CAR19-CAR20-T cells comprising PCTL153.

FIG. 7 shows the schematic diagram of the dosage regimen described in the validation test of the in-vivo tumor inhibitory activity of CAR19-CAR20-T cells comprising PCTL152 and CAR19-CAR20-T cells comprising PCTL153 in mice.

FIG. 8 shows the survival rates of the tumor-bearing mice in Group G1, Group G3 and Group G4 after being respectively administered with PBS, a vector that comprises the nucleic acid molecule encoding PCTL152 CAR19-CAR20, and a vector that comprises the nucleic acid molecule encoding PCTL153 CAR19-CAR20.

FIG. 9 shows the schematic diagram of the dosage regimen described in the validation test of the in-vivo tumor inhibitory activity of CAR19-CAR20-T cells comprising PCTL153, single-targeting CAR19-T cells, single-targeting CAR20-T cells and tandem CAR20-19-T cells in mice.

FIG. 10 shows the survival rates of the tumor-bearing mice in Group G1, Group G3, Group G4, Group G5 and Group G7 after being respectively administered with PBS, a vector that comprises the nucleic acid molecule encoding CAR-19, a vector that comprises the nucleic acid molecule encoding CAR-20, a vector that comprises the nucleic acid molecule encoding the combinational bispecific chimeric antigen receptor PCTL153 CAR19-CAR20, and a vector that comprises the nucleic acid molecule encoding the tandem bispecific chimeric antigen receptor CAR20-19.

FIG. 11 shows the killing efficiency of 7 kinds of CAR19-CAR22-T cells comprising bispecific chimeric antigen receptors with different structures for CD19⁺K562-luc-GFP target cells.

FIG. 12 shows the killing efficiency of 7 kinds of CAR19-CAR22-T cells comprising bispecific chimeric antigen receptors with different structures for CD22⁺K562-luc-GFP target cells.

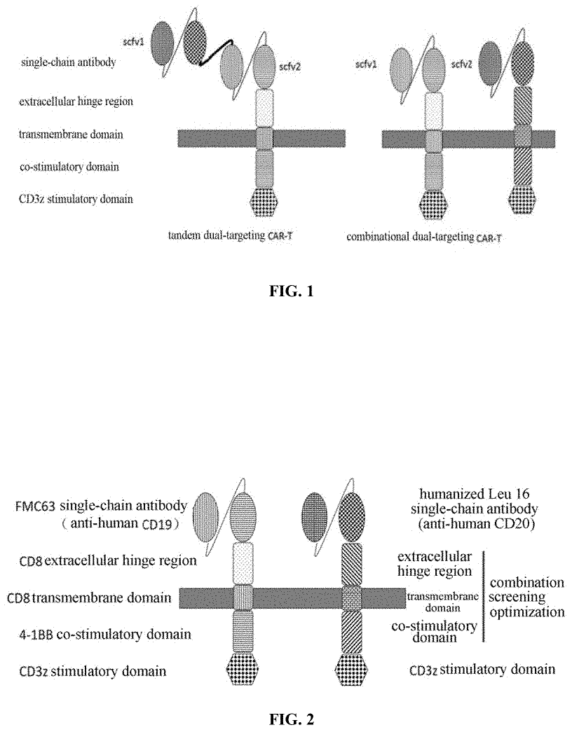

### DETAILED DESCRIPTION

The technical solutions of the present disclosure are further illustrated below by means of specific embodiments. It should be emphasized that the present disclosure is not limited to the specific embodiments exemplified and illustrated. In addition, titles of any section used herein are merely for purpose of organization, and are not to be construed as limiting the subject matters described.

Unless otherwise defined herein, scientific and technical terms used in the present disclosure will have the meanings commonly understood by one of ordinary skill in the art. In addition, unless otherwise required in the context, the terms in singular form should include the plural form thereof, and the terms in plural form should include the singular form thereof.

More specifically, as used in this specification and the appended claims, the singular forms “a”, “an” and “the” include plural referents unless the context clearly dictates otherwise. In the present application, unless otherwise indicated, “or” is used to denote “and/or”. In addition, the use of the term “comprising” and other forms (such as “including” and “containing”) is not restrictive. In addition, the ranges provided in the specification and the appended claims include the endpoints and all values between the endpoints.

### Example 1: Design of Combinational Bispecific Chimeric Antigen Receptors CAR19-CAR20 and CAR19-CAR22

The inventors designed 7 kinds of different CAR19-CAR20 combinational bispecific chimeric antigen receptors, wherein the structure of CAR19 was kept constant, that is, CAR19 with a structure of FMC63-CD8 hinge-CD8 TM-4-1BB-CD3z was selected (please refer to CN105392888A) and was combined with 7 kinds of CAR20s with different structures. Among them, the amino acid sequence of the antigen binding domain FMC63 in the above-mentioned CAR19 was as set forth in SEQ ID NO: 5, and the nucleotide sequence encoding this amino acid sequence was as set forth in SEQ ID NO: 6. The amino acid sequences of CD8 hinge, CD8 TM, 4-1BB and CD3z in the above-mentioned CAR19 were as set forth in SEQ ID NO: 9, SEQ ID NO: 11, SEQ ID NO: 13 and SEQ ID NO: 15, respectively; and the nucleotide sequences encoding the above-mentioned amino acid sequences were as set forth in SEQ ID NO: 10, SEQ ID NO: 12, SEQ ID NO: 14 and SEQ ID NO: 16.

The seven different CAR20s included Leu16-CD8 hinge-CD8 TM-4-1BBCSD-CD3z (the bispecific chimeric antigen receptor comprising both this CAR20 and the above-mentioned CAR19 was referred to as PCTL126), Leu16-CD28 hinge-CD28 TM-CD28CSD-CD3z (the bispecific chimeric antigen receptor comprising both this CAR20 and the above-mentioned CAR19 was referred to as PCTL137), Leu16-ICOS hinge-ICOSTM-ICOSCSD-CD3z (the bispecific chimeric antigen receptor comprising both this CAR20 and the above-mentioned CAR19 was referred to as PCTL138), Leu16-CD28 hinge-CD28 TM-OX40CSD-CD3z (the bispecific chimeric antigen receptor comprising both this CAR20 and the above-mentioned CAR19 was referred to as PCTL139), Leu16-IgG4mt10+N297A hinge-CD8 TM-4-1BBCSD-CD3z (the bispecific chimeric antigen receptor comprising both this CAR20 and the above-mentioned CAR19 was referred to as PCTL151), Leu16-IgG4mt10+N297A hinge-CD28 TM-CD28CSD-CD3z (the bispecific chimeric antigen receptor comprising both this CAR20 and the above-mentioned CAR19 was referred to as PCTL152), Leu16-IgG4mt10+N297A hinge-ICOSTM-ICOSCSD-CD3z (the bispecific chimeric antigen receptor comprising both this CAR20 and the above-mentioned CAR19 was referred to as PCTL153). The compositions of the seven different CAR20s were as shown in Table 1.

TABLE 1
Compositions of seven different CAR20s

| | | PCTL126 | PCTL137 | PCTL138 | PCTL139 | PCTL151 | PCTL152 | PCTL153 |
|---|---|---|---|---|---|---|---|---|
| Antigen binding | scFv | Leu16 | Leu16 | Leu16 | Leu16 | Leu16 | Leu16 | Leu16 |
| domain | | | | | | | | |
| Hinge region | Hinge | CD8 | CD28 | ICOS | CD28 | IgG4mt10 + | IgG4mt10 + | IgG4mt10 + |
| | | | | | | N297A | N297A | N297A |
| Transmembrane | Transmembrane | CD8 | CD28 | ICOS | CD28 | CD8 | CD28 | ICOS |
| domain | | | | | | | | |
| Co-stimulatory | costimulatory | 4-1BB | CD28 | ICOS | OX40 | 4-1BB | CD28 | ICOS |
| domain | domain | | | | | | | |
| Intracellular | signaling domain | CD3z | CD3z | CD3z | CD3z | CD3z | CD3z | CD3z |
| signaling domain | | | | | | | | |

To be specific, all the scFvs in the seven different CAR20s in Table 1 were a murine scFv (wherein the amino acid sequence of the murine scFv was as set forth in SEQ ID NO: 1, and the nucleotide sequence encoding this amino acid sequence was as set forth in SEQ ID NO: 2) humanized by conventional molecular biological means, and the humanized scFv was named Leu16, the amino acid sequence of which was as set forth in SEQ ID NO:3, and the nucleotide sequence encoding this amino acid sequence was as set forth in SEQ ID NO: 4.

There were four different options for the hinge region of the CAR20, i.e., CD8hinge, CD28hinge, ICOShinge or IgG4mt10+N297Ahinge, their amino acid sequences were as set forth in SEQ ID NO: 9, SEQ ID NO: 17, SEQ ID NO: 23 and SEQ ID NO: 31, respectively; and the nucleotide sequences encoding the above-mentioned amino acid sequences were as set forth in SEQ ID NO: 10, SEQ ID NO: 18, SEQ ID NO: 24 and SEQ ID NO: 32, respectively. Among them, the hinge region as shown by IgG4mt10+N297Ahinge was a hinge region that was derived from natural IgG4 and had 8 mutated amino acids. To be specific, in natural IgG4, the amino acid S at position 228 was substituted with P, the amino acid E at position 233 was substituted with P, the amino acid F at position 234 was substituted with V, the amino acid L at position 235 was substituted with A, the amino acid D at position 265 was substituted with A, the amino acid N at position 297 was substituted with A, the amino acid L at position 309 was substituted with V, and the amino acid R at position 409 was substituted with K, so as to deprive the binding ability of FcγR (Fc gamma receptor), avoid the antibody-dependent cell-mediated cytotoxicity (ADCC) and the complement-dependent cytotoxicity (CDC), thereby effectively enhancing the in-vivo activity of CAR-T cells.

There were three different options for the transmembrane domain of the CAR20, i.e., CD8TM, CD28TM or ICOSTM, their amino acid sequences were as set forth in SEQ ID NO: 11, SEQ ID NO: 19 and SEQ ID NO: 25, respectively; and the nucleotide sequences encoding the above-mentioned amino acid sequences were as set forth in SEQ ID NO: 12, SEQ ID NO: 20 [symbol] SEQ ID NO: 26, respectively.

There were four different options for the co-stimulator domain of the CAR20, i.e., 4-1BBCSD, CD28CSD, ICOSCSD and OX40CSD, their amino acid sequences were as set forth in SEQ ID NO: 13, SEQ ID NO: 21, SEQ ID NO: 27 and SEQ ID NO: 29, respectively; and the nucleotide sequences encoding the above-mentioned amino acid sequences were as set forth in SEQ ID NO: 14, SEQ ID NO: 22, SEQ ID NO: 28 and SEQ ID NO: 30, respectively. That is, the CAR20s in seven bispecific chimeric antigen receptor CAR19-CAR20s encoded the same antigen binding domain (i.e., having the same scFv) and CD3z signaling domain, wherein the amino acid sequence of the CD3z signaling domain was as set forth in SEQ ID NO: 15 (the nucleotide sequence encoding this amino acid sequence was as set forth in SEQ ID NO: 16). The only difference between the seven constructs was the different combinations of the hinge region, the transmembrane domain and the co-stimulator domain in CAR20. The schematic diagram of the structures of 7 kinds of CAR19-CAR20 combinational bispecific chimeric antigen receptors was as shown in FIG. 2.

The inventors also designed 7 kinds of different CAR19-CAR22 combinational bispecific chimeric antigen receptors, wherein the structure of CAR19 was kept constant, that is, CAR19 with a structure of FMC63-CD8 hinge-CD8 TM-4-1BB-CD3z was selected and was combined with 7 kinds of CAR22s with different structures. The seven different CAR22 included M971-CD8 hinge-CD8 TM-4-1BBCSD-CD3z (the bispecific chimeric antigen receptor comprising both this CAR22 and the above-mentioned CAR19 was referred to as PCTL81), M971-CD28 hinge-CD28 TM-CD28CSD-CD3z (the bispecific chimeric antigen receptor comprising both this CAR22 and the above-mentioned CAR19 was referred to as PCTL103), M971-ICOS hinge-ICOSTM-ICOSCSD-CD3z (the bispecific chimeric antigen receptor comprising both this CAR22 and the above-mentioned CAR19 was referred to as PCTL105), M971-CD28 hinge-CD28 TM-OX40CSD-CD3z (the bispecific chimeric antigen receptor comprising both this CAR22 and the above-mentioned CAR19 was referred to as PCTL124), M971-IgG4mt10+N297A hinge-CD8 TM-4-1BBCSD-CD3z (the bispecific chimeric antigen receptor comprising both this CAR22 and the above-mentioned CAR19 was referred to as PCTL148), M971-IgG4mt10+N297A hinge-CD28 TM-CD28CSD-CD3z (the bispecific chimeric antigen receptor comprising both this CAR22 and the above-mentioned CAR19 was referred to as PCTL149), and M971-IgG4mt10+N297A hinge-ICOSTM-ICOSCSD-CD3z (the bispecific chimeric antigen receptor comprising both this CAR22 and the above-mentioned CAR19 was referred to as PCTL150). The compositions of the seven different CAR22s were as shown in Table 2.

TABLE 2
Compositions of seven different CAR22s

| | | PCTL181 | PCTL103 | PCTL105 | PCTL124 | PCTL148 | PCTL149 | PCTL150 |
|---|---|---|---|---|---|---|---|---|
| Antigen binding | scFv | M971 | M971 | M971 | M971 | M971 | M971 | M971 |
| domain | | | | | | | | |
| Hinge region | Hinge | CD8 | CD28 | ICOS | CD28 | IgG4mt10 + | IgG4mt10 + | IgG4mt10 + |
| | | | | | | N297A | N297A | N297A |
| Transmembrane | Transmembrane | CD8 | CD28 | ICOS | CD28 | CD8 | CD28 | ICOS |
| domain | | | | | | | | |
| Co-stimulatory | costimulatory | 4-1BB | CD28 | ICOS | OX40 | 4-1BB | CD28 | ICOS |
| domain | domain | | | | | | | |
| Intracellular | signaling domain | CD3z | CD3z | CD3z | CD3z | CD3z | CD3z | CD3z |
| signaling domain | | | | | | | | |

To be specific, the seven different CAR22s in Table 2 had an scFv with an amino acid sequence as set forth in SEQ ID NO: 7, and the nucleotide sequence encoding this amino acid sequence was as set forth in SEQ ID NO: 8. There were four different options for the hinge region of the CAR22, i.e., CD8hinge, CD28hinge, ICOShinge or IgG4mt10+N297Ahinge, their amino acid sequences were as set forth in SEQ ID NO: 9, SEQ ID NO: 17, SEQ ID NO: 23 and SEQ ID NO: 31, respectively; and the nucleotide sequences encoding the above-mentioned amino acid sequences were as set forth in SEQ ID NO: 10, SEQ ID NO: 18, SEQ ID NO: 24 and SEQ ID NO: 32, respectively. Among them, the hinge region as shown by IgG4mt10+N297A was a hinge region that was derived from natural IgG4 and has 8 mutated amino acids. To be specific, in natural IgG4, the amino acid S at position 228 was substituted with P, the amino acid E at position 233 was substituted with P, the amino acid F at position 234 was substituted with V, the amino acid L at position 235 was substituted with A, the amino acid D at position 265 was substituted with A, the amino acid N at position 297 was substituted with A, the amino acid L at position 309 was substituted with V, the amino acid R at position 409 was substituted with K, so as to deprive the binding ability of FcγR (Fc gamma receptor), avoid the antibody-dependent cell-mediated cytotoxicity (ADCC) and the complement-dependent cytotoxicity (CDC), thereby effectively enhancing the in-vivo activity of CAR-T cells.

There were three different options for the transmembrane domain of the CAR22, i.e., CD8TM, CD28TM or ICOSTM, their amino acid sequences were as set forth in SEQ ID NO: 11, SEQ ID NO: 19 and SEQ ID NO: 25, respectively; and the nucleotide sequences encoding the above-mentioned amino acid sequences were as set forth in SEQ ID NO: 12, SEQ ID NO: 20 and SEQ ID NO: 26, respectively.

There were four different options for the co-stimulator domain of the CAR22, i.e, 4-1BBCSD, CD28CSD, ICOSCSD and OX40CSD, their amino acid sequences were as set forth in SEQ ID NO: 13, SEQ ID NO: 21, SEQ ID NO: 27 and SEQ ID NO: 29, respectively; and the nucleotide sequences encoding the above-mentioned amino acid sequences were as set forth in SEQ ID NO: 14, SEQ ID NO: 22, SEQ ID NO: 28 and SEQ ID NO: 30, respectively. That is, the CAR22s in seven bispecific chimeric antigen receptor CAR19-CAR22s encoded the same antigen binding domain (i.e., having the same scFv) and CD3z signaling domain, wherein the amino acid sequence of the CD3z signaling domain was as set forth in SEQ ID NO: 15 (the nucleotide sequence encoding this amino acid sequence was as set forth in SEQ ID NO: 16). The only difference between the seven constructs was the different combinations of the hinge region, the transmembrane domain and the co-stimulator domain in CAR22s.

### Example 2: Comparison of the Killing Effects of CAR19-CAR20-T Cells Prepared from Bispecific Chimeric Antigen Receptors with Different Structures on Target Cells

The CAR19-CAR20 combinational bispecific chimeric antigen receptors as described in Example 1 were used to prepare dual-targeting CAR-T cells, and then the dual-targeting CAR-T cells were co-incubated with two different kinds of target cells, i.e., CD19⁺K562-luc-GFP and CD20⁺K562-luc-GFP for 18 to 24 hours at different effector cell (E): target cell (T) ratios, that is, co-incubated at a E/T ratio of 1:1, 2.5:1, 5:1, 10:1 or 20:1, respectively. T cells without genetic modification (that is, T cells that had not been subjected to lentivirus infection, hereinafter referred to as NC-T cells) were used as the background control, the constructed target cell strain was labeled with luciferase, and the killing effects of effector cells on target cells were determined based on the principle of chemiluminescence. The specific operations were as follows.

(1) Isolation of PBMC from Peripheral Blood, Isolation and Activation of T Cells, Lentiviral Transduction and In-Vitro Culture

Healthy donors tested negative for HBV, HCV and HIV were selected, 100 ml of blood was drawn from the median cubital vein, PBMCs were isolated from buffy coat via Ficoll density gradient centrifugation, and the number of CD3⁺T cells were calculated according to the percentage of CD3⁺T cells determined via whole blood flow cytometry. The magnetic beads were aspirated in its using amount (DynaBeads CD3/CD28:CD3⁺T cell=3:1) and incubated with cells in the buffy coat for 30 min. CD3⁺T cells were isolated and activated by Dynabeads CD3/CD28 (Lifetechnologies, Cat. No.: 40203D) for 24 hours, followed by the determination of the proportion of CD25⁺CD69⁺ T cells via flow cytometry (the proportion of CD25⁺CD69⁺ T cell: 71%). CD3⁺ T cells were subjected to lentiviral transduction after activation. A Novonectin-coated 24-well plate was incubated at 37° C. for 2 hours, the cell suspensions obtained after the above operations were respectively formulated into transduction systems with each of the prepared lentiviruses (that is, lentiviruses respectively comprising PCTL126, PCTL137, PCTL138, PCTL139, PCTL151, PCTL152, and PCTL153) (MOI=8), Synperonic® F108 (Sigma, Cat. No.: 07579-250G-F, 10 μg/ml) and Tscm (2 U/ml), the transduction systems were charged in the coated 24-well plate, the cell density was adjusted to 1.0E+06 cells/ml, followed by centrifugation at 500 g for 30 min and subsequent static culture in an incubator containing CO₂ at 37° C. for 48 h. After transfection, cells were cultured in X-vivo15 medium (LONZA, Cat. No.: 04-418Q) containing 5% FBS, Tscm (final concentration: 2 U/ml) was supplemented every other day, cell counting was conducted, the cell density was adjusted to 0.5E+06 cells/ml, and cells were harvested after being cultured to Day 8 to Day 10.

(2) Preparation of effector cells (dual-targeting CAR-T cells): (NC-T cells (T cells that had not been subjected to lentivirus infection) that had been proliferated for 5 to 7 days and CAR-T cells in each group were taken, followed by observation under a microscope to judge whether the growth status of cells was normal. @ NC-T cells and CAR-T cells in each group were collected into a 15-mL centrifuge tube or a 50-mL centrifuge tube, and the total number of cells was counted (Cellometer k2 cell counter). @ The collected cells were washed once or twice with sterile PBS (Hyclone, Cat. No.: SH30256.01) and centrifuged at 1500 rpm for 5 minutes at 25° C. @ The washed cell pellet was re-suspended with T cell culture medium X-VIVO15 (LONZA, Cat. No.: 04-418Q) (without autologous serum and IL-2), and the cell density was adjusted to 5.0E+07 cells/mL.

(3) Preparation of target cells: \textcircled{1} Target cells, i.e., CD19⁺K562-luc-GFP and CD20⁺K562-luc-GFP (Tsukahara et al. Biochem Biophys Res Commun. 2013; 438(1):84-89), were taken and observed under a microscope to judge whether the cell status was normal. \textcircled{2} The two kinds of target cells mentioned above were respectively collected into a 15-mL centrifuge tube or a 50-mL centrifuge tube, and the total number of cells was counted. \textcircled{3} The collected cells were washed once or twice with sterile PBS and centrifuged at for 5 minutes 1500 rpm at 25° C. \textcircled{4} The washed cell pellet was re-suspended with RPM11640 (gibco, Cat. No.: 11875-093) (without FBS), and the cell density was adjusted to 5.0E+06 cells/mL.

(4) In-vitro killing: \textcircled{1} Preparation of killing systems: In a 1.5-mL centrifuge tube, effector cells (i.e., NC-T cells and CAR-T cells in each group) with adjusted density were respectively mixed with target cells (i.e., CD19⁺K562-luc-GFP and CD20⁺K562-luc-GFP) at different effector-to-target ratios, specifically, effector cells (CAR-T cells) and target cells were respectively mixed at a ratio of 1:1, 2.5:1, 5:1, 10:1 or 20:1, and T cell culture medium X-VIVO15 (LONZA, Cat. No.: 04-418Q) (without autologous serum and IL-2) was added until the total volume was up to 200 μL; \textcircled{2} 200-μL killing systems prepared above were respectively transferred into a 96-well V-shape plate for co-incubation for 24 hours. \textcircled{3} After 24 hours, cells in each well of the 96-well V-shape plate were gently pipetted and mixed evenly, and 100 μL of cell suspensions were respectively transferred into a 96-well plate with white wall and non-transparent bottom. 100 μL of ONE-Glo™ Luciferase Assay Substrate was added, and chemiluminescence (Luminescence) was determined by Luminoskan Ascent chemiluminescence analyzer after the system was incubated in the dark at room temperature for 10 minutes.

Calculation of killing efficiency: Killing efficiency=(the corresponding value of NC-T cells−the value of specific CAR19-CAR20-T cell at corresponding effector-to-target ratio)/the corresponding value of NC-T cells

Experimental results: As could be seen from the results as shown in Table 3, in a case where the effector-to-target ratio was 10:1, CAR19-CAR20-T cells comprising one of the seven bispecific chimeric antigen receptors (that is, PCTL126, PCTL137, PCTL138, PCTL139, PCTL151, PCTL152 and PCTL153) had a killing efficiency of more than 90% for CD19+K562-luc-GFP target cells. Among them, CAR19-CAR20-T cells comprising PCTL152 and CAR19-CAR20-T cells comprising PCTL153 had a killing efficiency of approximately 100% for CD19⁺K562-luc-GFP target cells (please refer to Table 3 and FIG. 3 for details). In addition, CAR19-CAR20-T cells comprising PCTL152 and CAR19-CAR20-T cells comprising PCTL153 still had relatively high killing efficiency for the target cells in a case where the effector-to-target ratio was relatively low.

TABLE 3
Killing efficiency of bispecific chimeric antigen receptor
CAR19-CAR20-T cells for CD19⁺K562-luc-GFP target cells

| | 1:1 | 2.5:1 | 5:1 | 10:1 | 20:1 |
|---|---|---|---|---|---|
| PCTL137 | −273.22% | 40.27% | 94.13% | 96.58% | 98.60% |
| PCTL138 | −178.91% | 75.38% | 97.68% | 98.31% | 99.41% |
| PCTL139 | −153.56% | 41.80% | 96.77% | 97.60% | 98.72% |
| PCTL126 | −26.72% | 87.59% | 97.59% | 98.95% | 99.65% |
| PCTL151 | −28.95% | 83.53% | 93.95% | 96.44% | 99.07% |
| PCTL152 | 40.19% | 95.92% | 99.12% | 99.58% | 99.85% |
| PCTL153 | 43.95% | 96.83% | 97.71% | 99.40% | 99.72% |

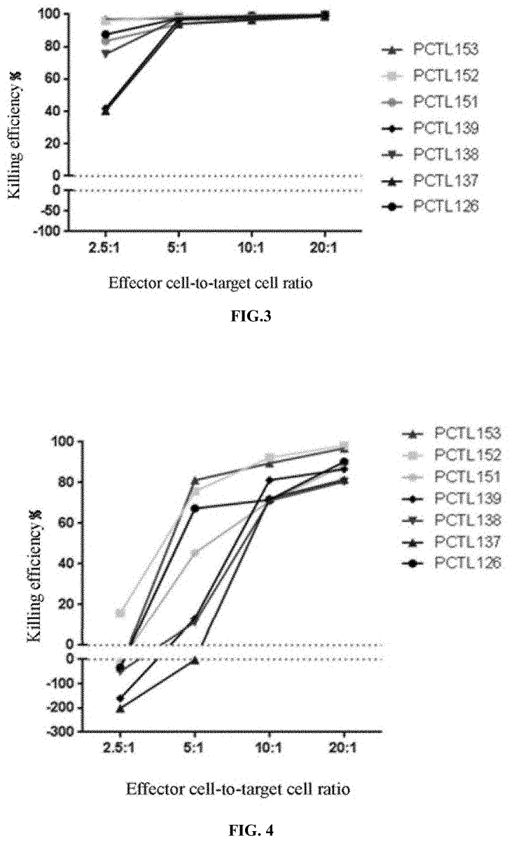

As could be seen from the results as shown in Table 4, in a case where the effector-to-target ratio was 20:1, CAR19-CAR20-T cells comprising one of the seven bispecific chimeric antigen receptors (that is, PCTL126, PCTL137, PCTL138, PCTL139, PCTL151, PCTL152 and PCTL153) had a killing efficiency of more than 80% for CD20⁺K562-luc-GFP target cells. Among these, CAR19-CAR20-T cells comprising PCTL152 and CAR19-CAR20-T cells comprising PCTL153 had a killing efficiency of approximately 100% for CD20⁺K562-luc-GFP target cells (please refer to Table 4 and FIG. 4 for details).

TABLE 4
Killing efficiency of bispecific chimeric antigen receptor
CAR19-CAR20-T cells for CD20⁺K562-luc-GFP target cells

| | 1:1 | 2.5:1 | 5:1 | 10:1 | 20:1 |
|---|---|---|---|---|---|
| PCTL137 | −269.38% | −201.37% | −2.41% | 72.28% | 81.53% |
| PCTL138 | −204.88% | −49.82% | 11.09% | 71.10% | 80.52% |
| PCTL139 | −279.06% | −160.74% | 13.09% | 81.29% | 86.59% |
| PCTL126 | −138.73% | −33.19% | 67.29% | 71.61% | 90.32% |
| PCTL151 | −136.73% | −12.02% | 45.50% | 70.90% | 88.66% |
| PCTL152 | −182.88% | 15.71% | 75.81% | 92.37% | 98.25% |
| PCTL153 | −161.64% | −32.69% | 81.21% | 89.58% | 96.97% |

### Example 3: T Cell Phenotypes Exhibited by CAR19-CAR20-T Cells Prepared from Bispecific Chimeric Antigen Receptors with Different Structures

Dual-targeting CAR-T cells were prepared using the CAR19-CAR20 combinational bispecific chimeric antigen receptors as described in Example 1, and differentiated cell populations were analyzed by a flow cytometer using conventional T cell differentiation antigens and antibodies on Day 7 to Day 10 after lentiviral transfection.

Experimental methods: 7 kinds of bispecific chimeric antigen receptor CAR19-CAR20s prepared in Example 1 were selected as experimental materials, and the corresponding dual-targeting CAR-T cells were prepared according to the preparation method of effector cells as described in Example 2. For each of the 7 kinds of dual-targeting CAR-T cells as prepared, 1×10⁶ CAR-T cells were taken, the CAR-T cells were washed with PBS and then incubated with CD62L-PE-Cy5 antibody (BD, Cat. No.: 555545) and CD45RO-FITC antibody (BD, Cat. No.: 555492) in a freezer at 4° C. for 30 min. After the completion of the incubation with antibodies, the resultant was washed with PBS (Hyclone, Cat. No.: SH30256.01) 2-3 times, re-suspended with 500 μl of PBS, and then placed in a flow cytometry tube to prepare for determination on a flow cytometer.

Experimental results: As shown in FIG. 5, the results indicated that all the analyzed dual-targeting CAR-T cells were capable of maintaining a relatively high proportion of stem cell-like central memory T cells (TSCMs). Studies had reported that the proportion of the stem cell-like central memory T cell population was closely related to the tumoricidal activity, proliferating ability and lasting immunological memory of CAR-T cells in organisms. This suggested that the dual-targeting CAR-T cells provided in the present disclosure were capable of having relatively good tumoricidal activity, proliferating ability and lasting immunological memory in organisms.

### Example 4: Detection of the Expression of CAR19 and CAR20 in CAR19-CAR20-T Cells Comprising PCTL152 and CAR19-CAR20-T Cells Comprising PCTL153

Experimental methods: CAR19-CAR20-T cells comprising PCTL152 and CAR19-CAR20-T cells comprising PCTL153 with relatively high killing efficiency in Example 2 were selected as experimental materials. For each of the two kinds of CAR-T cells mentioned above, 1×10⁶ CAR-T cells were taken, washed with 4% BSA (2500 rpm, 5 min) three times, and then incubated with antibodies as follows. (1) Cells were incubated with Alexa Fluor 647 AffiniPure Goat Anti-Human IgG (1:100 to 1:800) in a freezer at 4° C. for 30 min. After the completion of the incubation with the antibody, the resultant was washed with 4% BSA (2500 rpm, 5 min) three times, and then incubated with the following antibody, i.e., (2) PE-labeled CAR19 (iFMC63) idiotype (Qin et al. Mol Ther Oncolytics. 2018; 11: 127-137) (1 μg/ml) in a freezer at 4° C. for 30 min. After the completion of the incubation with the antibody, the resultant was washed with 4% BSA 2-3 times (2500 rpm, 5 min), re-suspended with 500 μl of PBS, and then placed in a flow cytometry tube to prepare for determination on a flow cytometer.

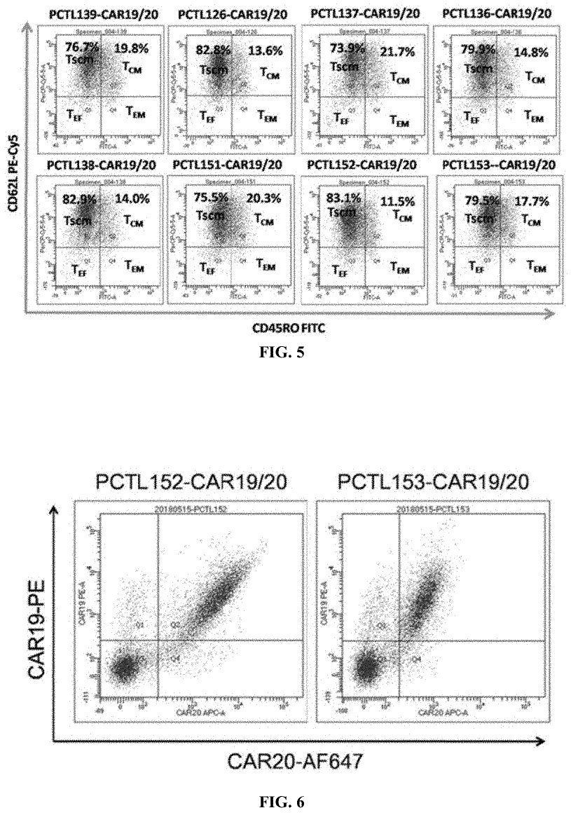

Experimental results: As shown in FIG. 6, both CAR19 protein and CAR20 protein could be simultaneously detected on the surface of T cells. The results indicated that the constructed CAR19-CAR20-T cells comprising PCTL152 and CAR19-CAR20-T cells comprising PCTL153 were capable of expressing CAR19⁺CAR20⁺ dual-positive population well.

### Example 5: Validation of the In-Vivo Tumor Inhibitory Activity of CAR19-CAR20-T Cells Comprising PCTL152 and CAR19-CAR20-T Cells Comprising PCTL153 in Mice

Experimental methods: CAR19-CAR20-T cells comprising PCTL152 and CAR19-CAR20-T cells comprising PCTL153 with relatively high killing efficiency in Example 2 were selected as the experimental groups, and PBS was selected as the control group. Raji-Luc cells (Biocytogen, Cat. No.: B-HCL-010) re-suspended with PBS were inoculated into B-NDG® (B-NSG) mice with a concentration of 5×10⁵ cells/0.2 mL and a volume of 0.2 mL/mice by intravenous injection via tail vein. On the day of inoculation, a small animal imaging system was utilized to observe whether the tumor inoculation was successful. On Day 3 after inoculation, tumor growth was measured by using the small animal imaging system. When the average imaging signal reached approximately 1×10⁶ [(P/S)/(cm²/sr)], 8 mice with moderate tumor imaging signal were selected and enrolled, and were randomly assigned to 3 groups (2 mice in Group G1, 3 mice in each of Group G3 and Group G4). Mice showing excessively strong/excessively weak fluorescence signal were excluded. Administration began on the day of grouping, and tumor growth (detected and recorded by the small animal imaging system) was detected and the body weights of the animals were measured on Day 4 after administration. Afterwards, mice were detected on the imaging system once a week (Day 4, Day 11, Day 18 and Day 25) and the body weights of the animals were measured twice a week. The specific dosage regimen was as shown in FIG. 7. Please refer to Table 5 for the specific details of the type of the administered substances, the number of mice and the dosage of administration in each group.

TABLE 5
Type of the administered substances, number of mice and dosage
of administration of Group G1, Group G3 and Group G4

| | Type of the | | |
|---|---|---|---|
| | administered | Number | |
| Group | substances | of mice | Dosage of administration |
| G1 | PBS | 2 | PBS 200 μl/mice |
| G3 | PCTL152 | 3 | Total T 0.1E+07/200 μl/mice |
| G4 | PCTL153 | 3 | Total T 0.1E+07/200 μl/mice |

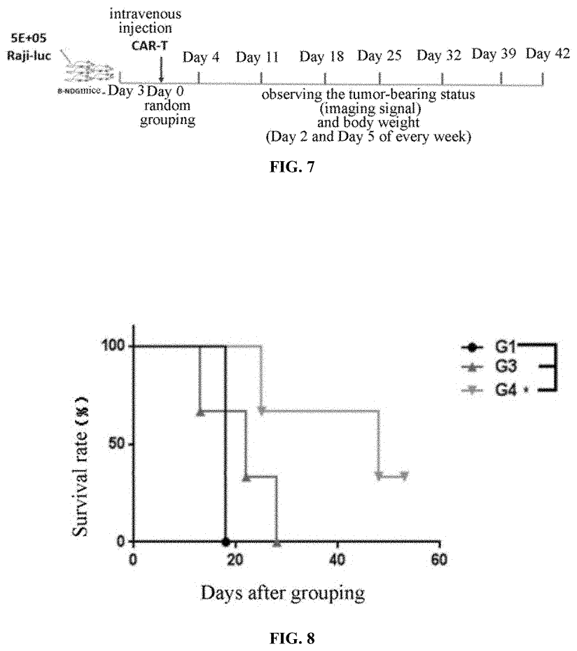

Experimental results: Up to Day 28, all the mice in Group G1 and Group G3 died, and the survival rate of the mice in Group G4 was 67.7%. It could be seen that, as compared with CAR19-CAR20-T cells comprising PCTL152, CAR19-CAR20-T cells comprising PCTL153 were capable of increasing the survival rate of the tumor-bearing mice significantly (please refer to FIG. 8).

### Example 6: Validation of the In-Vivo Tumor Inhibitory Activity of CAR19-CAR20-T Cells Comprising PCTL153, Single-Targeting CAR19-T Cells, Single-Targeting CAR20-T Cells and Tandem CAR20-19-T Cells in Mice

Experimental methods: Raji-Luc cells re-suspended with PBS were inoculated into B-NDG® (B-NSG) mice with a concentration of 5×10⁵ cells/0.2 mL and a volume of 0.2 mL/mice by intravenous injection via tail vein, and 54 mice were inoculated in total. On the day of inoculation, a small animal imaging system was utilized to observe whether the tumor inoculation was successful. Tumor growth was measured by using the small animal imaging system after the successful inoculation. When the average imaging signal reached approximately 1×10⁶[(P/S)/(cm²/sr)], 30 mice with moderate tumor imaging signal were selected and enrolled, and were randomly assigned to 5 groups (6 mice per group). Tumor-bearing mice showing excessively strong/excessively weak living imaging signal were excluded. Administration began on the day of grouping, the body weights of the experimental animals and the tumor growth (detected and recorded by the small animal imaging system) were continuously observed after administration. Tumor growth was measured on Day 4, Day 7 and Day 11 after grouping, afterwards, tumor growth was measured once a week (detected and recorded by the small animal imaging system). The body weights of the animals were measured twice a week, clinical observation was performed, and the measured values were recorded. The specific dosage regimen was as shown in FIG. 9. Please refer to Table 6 for the specific details of the type of the administered substances, the number of mice and the dosage of administration.

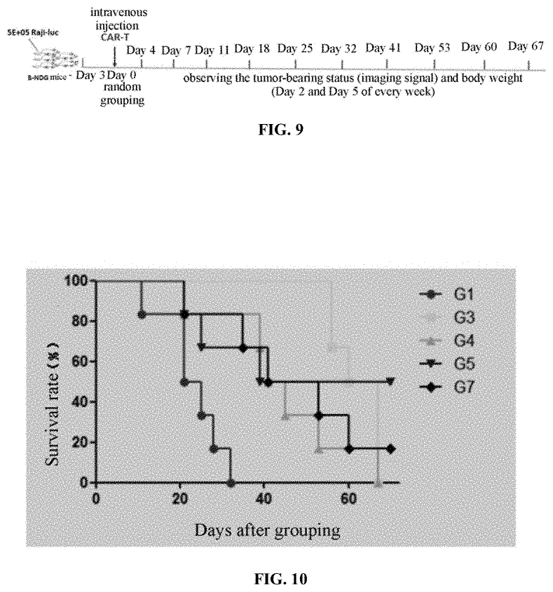

Experimental results: As compared with the traditional single-targeting CAR19-T cells (Group G3, the structure and sequence of the CAR19 comprised therein were the same as those of the CAR19 described in Example 1 of the present disclosure, i.e., FMC63-CD8 hinge-CD8 TM-4-1BB-CD3z), single-targeting CAR20-T cells (Group G4, the structure and sequence of the CAR20 comprised therein were the same as those of the CAR20 in the PCTL153 described in Example 1 of the present disclosure, i.e., Leu16-IgG4mt10+N297A hinge-ICOSTM-ICOSCSD-CD3z) and tandem CAR20-19-T cells (Group G7, CD20 scFv-(EAAAK)3-CD19 scFv-IgG4 hinge-CD28 TM-4-1BB-CD3z, please refer to Zah et al. Cancer Immunol Res. 2016; 4(6):498-508 for details), the combinational dual-targeting CAR19-CAR20-T cells comprising PCTL153 (Group G5) were capable of increasing the survival rate of tumor-bearing mice significantly (please refer to FIG. 10).

TABLE 6
Type of the administered substances, number of
mice and dosage of administration of Group G1,
Group G3, Group G4, Group G5 and Group G7

| | Type of the | | |
|---|---|---|---|
| | administered | Number | |
| Group | substances | of mice | Dosage of administration |
| G1 | PBS | 6 | PBS 200 μl/mice |
| G3 | CAR-19-T | 6 | Total T 1.0E+07/200 μl/mice |
| G4 | CAR-20-T | 6 | Total T 1.0E+07/200 μl/mice |
| G5 | PCTL153 | 6 | Total T 1.0E+07/200 μl/mice |
| | (combinational | | |
| | CAR19-CAR20) | | |
| G7 | tandem CAR20- | 6 | Total T 1.0E+07/200 μl/mice |
| | CAR19 | | |

### Example 7: Comparison of the Killing Effects of CAR19-CAR22-T Cells Prepared from Bispecific Chimeric Antigen Receptors with Different Structures on Target Cells

The combinational bispecific chimeric antigen receptor CAR19-CAR22s as described in Example 1 were used to prepare dual-targeting CAR-T cells, and then the dual-targeting CAR-T cells were co-incubated with two different kinds of target cells, i.e., CD19⁺K562-luc-GFP and CD22⁺K562-luc-GFP for 18 to 24 hours at different effector cell (E): target cell (T) ratios, that is, at a E/T ratio of 5:1, 10:1 or 20:1, respectively. T cells without genetic modification (that is, T cells that had not been subjected to lentivirus infection, hereinafter referred to as NC-T cells) were used as the background control, the constructed target cell strain was labeled with luciferase, and the killing effects of effector cells on target cells were determined based on the principle of chemiluminescence. The specific operations were as follows.

(1) Isolation of PBMC from peripheral blood, isolation and activation of T cells, lentiviral transduction and in-vitro culture

Healthy donors tested negative for HBV, HCV and HIV were selected, 100 ml of blood was drawn from the median cubital vein, PBMCs were isolated from buffy coat via Ficoll density gradient centrifugation, and the number of CD3⁺T cells were calculated according to the percentage of CD3⁺ T cells determined via whole blood flow cytometry. The magnetic beads were aspirated in its using amount (DynaBeads CD3/CD28:CD3⁺ T cell=3:1) and incubated with cells in the buffy coat for 30 min. CD3*T cells were isolated and activated by Dynabeads CD3/CD28 (Lifetechnologies, Cat. No.: 40203D) for 24 hours, followed by the determination of the proportion of CD25⁺CD69⁺ T cells via flow cytometry (the proportion of CD25⁺CD69⁺ T cell: 71%). CD3⁺ T cells were subjected to lentiviral transduction after activation. A Novonectin-coated 24-well plate was incubated at 37° C. for 2 hours, the cell suspensions obtained after the above operations were respectively formulated into transduction systems with each of the prepared lentiviruses (that is, lentiviruses respectively comprising PCTL81, PCTL103, PCTL105, PCTL124, PCTL148, PCTL149, and PCTL150) (MOI=8), Synperonic® F108 (Sigma, Cat. No.: 07579-250G-F, 10 μg/ml) and Tscm (2 U/ml), the transduction systems were charged in the coated 24-well plate, the cell density was adjusted to 1.0E+06 cells/ml, followed by centrifugation at 500 g for 30 min and subsequent static culture in an incubator containing CO₂ at 37° C. for 48 h. After transfection, cells were cultured in X-vivo15 medium (LONZA, Cat. No.: 04-418Q) containing 5% FBS, Tscm (final concentration: 2 U/ml) was supplemented every other day, cell counting was conducted, the cell density was adjusted to 0.5E+06 cells/ml, and cells were harvested after being cultured to Day 8 to Day 10.

(2) Preparation of effector cells (dual-targeting CAR-T cells): \textcircled{1} NC-T cells (T cells that had not been subjected to lentivirus infection) that had been proliferated for 5 to 7 days and CAR-T cells in each group were taken, followed by observation under a microscope to judge whether the growth status of cells was normal. \textcircled{2} NC-T cells and CAR-T cells in each group were collected into a 15-mL centrifuge tube or a 50-mL centrifuge tube, and the total number of cells was counted (Cellometer k2 cell counter). \textcircled{3} The collected cells were washed once or twice with sterile PBS (Hyclone, Cat. No.: SH30256.01) and centrifuged at 1500 rpm for 5 minutes at 25° C. \textcircled{4} The washed cell pellet was re-suspended with T cell culture medium X-VIVO15 (LONZA, Cat. No.: 04-418Q) (without autologous serum and IL-2), and the cell density was adjusted to 5.0E+07 cells/mL.

(3) Preparation of target cells: \textcircled{1} Target cells, i.e., CD19⁺K562-luc-GFP and CD22⁺K562-luc-GFP (Tsukahara et al. Biochem Biophys Res Commun. 2013; 438(1):84-89), were taken and observed under a microscope to judge whether the cell status was normal. \textcircled{2} The two kinds of target cells mentioned above were respectively collected into a 15-mL centrifuge tube or a 50-mL centrifuge tube, and the total number of cells was counted. \textcircled{3} The collected cells were washed once or twice with sterile PBS and centrifuged at 1500 rpm for 5 minutes at 25° C. \textcircled{4} The washed cell pellet was re-suspended with RPMI1640 (gibco, Cat. No.: 11875-093) (without FBS), and the cell density was adjusted to 5.0E+06 cells/mL.

(4) In-vitro killing: \textcircled{1} Preparation of killing systems: In a 1.5-mL centrifuge tube, effector cells (i.e., NC-T cells and CAR-T cells in each group) with adjusted density were respectively mixed with target cells (i.e., CD19⁺K562-luc-GFP and CD22⁺K562-luc-GFP) at different effector-to-target ratios, specifically, effector cells (CAR-T cells) and target cells were respectively mixed at a ratio of 5:1, 10:1 or 20:1, and T cell culture medium X-VIV015 (LONZA, Cat. No.: 04-418Q) (without autologous serum and IL-2) was added until the total volume was up to 200 μL. \textcircled{2} 200-μL killing systems prepared above were respectively transferred into a 96-well V-shape plate for co-incubation for 24 hours. \textcircled{3} After 24 hours, cells in each well of the 96-well V-shape plate were gently pipetted and mixed evenly, and 100 μL of cell suspensions were respectively transferred into a 96-well plate with white wall and non-transparent bottom. 100 μL of ONE-Glo™ Luciferase Assay Substrate was added, and chemiluminescence (Luminescence) was determined by Luminoskan Ascent chemiluminescence analyzer after the system was incubated in the dark at room temperature for 10 minutes.

Calculation of killing efficiency: Killing efficiency=(the corresponding value of NC-T cells−the value of specific CAR19-CAR22-T cell at corresponding effector-to-target ratio)/the corresponding value of NC-T cell

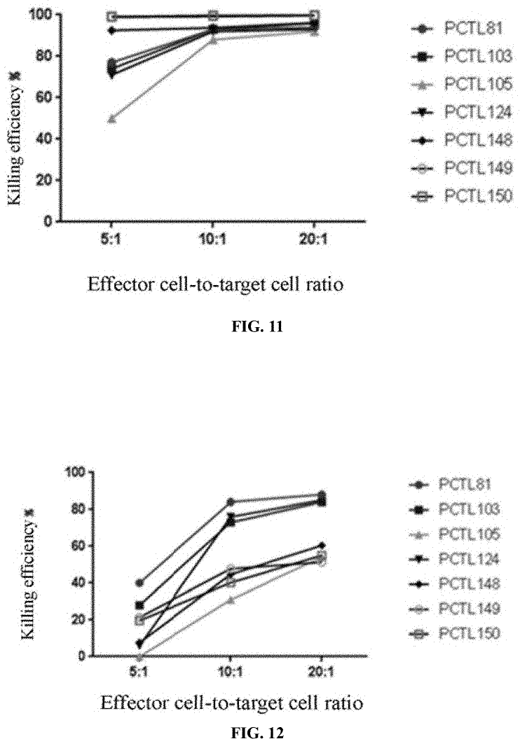

Experimental results: As could be seen from the results as shown in FIG. 11, in a case where the effector-to-target ratio was 10:1, CAR19-CAR22-T cells comprising one of the seven bispecific chimeric antigen receptors (that is, PCTL81, PCTL103, PCTL105, PCTL124, PCTL148, PCTL149 and PCTL150) had a killing efficiency of more than 90% for CD19⁺K562-luc-GFP target cells (please refer to FIG. 11 for details). As could be seen from the results as shown in FIG. 12, all the CAR19-CAR22-T cells comprising one of the seven bispecific chimeric antigen receptors (that is, PCTL81, PCTL103, PCTL105, PCTL124, PCTL148, PCTL149 and PCTL150) had killing effects on CD22⁺K562-luc-GFP target cells and were apparently effector-to-target ratio-dependent (please refer to FIG. 12 for details).

1. A chimeric antigen receptor composed of an antigen binding domain, an extracellular hinge region, a transmembrane domain, a co-stimulatory domain and a CD3z signaling domain.

2. The chimeric antigen receptor according to claim 1, wherein
the extracellular hinge region is any one selected from the group consisting of CD8 extracellular hinge region (CD8hinge), CD28 extracellular hinge region (CD28hinge), ICOS extracellular hinge region (ICOShinge) and IgG4mt10+N297A extracellular hinge region (IgG4mt10+N297Ahinge);
the transmembrane domain is any one selected from the group consisting of CD8 transmembrane domain (CD8TM), CD28 transmembrane domain (CD28TM) and ICOS transmembrane domain (ICOSTM); and
the co-stimulatory domain is any one selected from the group consisting of 4-1BB co-stimulatory domain (4-1BBCSD), CD28 co-stimulatory domain (CD28CSD), ICOS co-stimulatory domain (ICOSCSD) and OX40 co-stimulatory domain (OX40CSD).

3. The chimeric antigen receptor according to claim 2, wherein structures comprising the extracellular hinge region, the transmembrane domain and the co-stimulatory domain are respectively as follows: CD8hinge-CD8TM-4-1BBCSD, CD28hinge-CD28TM-CD28CSD, ICOShinge-ICOSTM-ICOSCSD, CD28hinge-CD28TM-OX40CSD, IgG4mt10+N297Ahinge-CD8TM-4-1BBCSD, IgG4mt10+N297Ahinge-CD28 TM-CD28CSD or IgG4mt10⁺N297Ahinge-ICOSTM-ICOSCSD.

4. The chimeric antigen receptor according to claim 3, wherein
an amino acid sequence of the IgG4mt10+N297Ahinge-CD8TM-4-1BBCSD is SEQ ID NO: 36;
an amino acid sequence of the IgG4mt10+N297Ahinge-CD28TM-CD28CSD is SEQ ID NO: 37; and
an amino acid sequence of the IgG4mt10+N297Ahinge-ICOSTM-ICOSCSD is SEQ ID NO: 38.

5. The chimeric antigen receptor according to claim 2, wherein the antigen binding domain is a single-chain antibody (scFv) or a single domain antibody (sdAb).

6. The chimeric antigen receptor according to claim 5, wherein the antigen binding domain recognizes CD20 or recognizes CD22.

7. The chimeric antigen receptor according to claim 6, wherein the antigen binding domain is Leu16, said Leu16 is a humanized scFv recognizing CD20 and an amino acid sequence of said Leu16 is as set forth in SEQ ID NO: 3.

8. The chimeric antigen receptor according to claim 6, wherein the antigen binding domain is M971, said M971 is an scFv recognizing CD22 and an amino acid sequence of said M971 is as set forth in SEQ ID NO: 7.

9. A chimeric antigen receptor T cell (CAR-T cell), wherein the CAR-T cell expresses the chimeric antigen receptor of claim 1.

10. The chimeric antigen receptor T cell (CAR-T cell) according to claim 9, wherein the CAR-T cell expresses two chimeric antigen receptors comprising different antigen binding domains.

11. The CAR-T cell according to claim 10, wherein the two independent chimeric antigen receptors are CAR19 and CAR20, respectively; wherein said CAR19 recognizes CD19, and said CAR20 recognizes CD20.

12. The CAR-T cell according to claim 10, wherein the two independent chimeric antigen receptors are CAR19 and CAR22, respectively; wherein said CAR19 recognizes CD19, and said CAR22 recognizes CD22.

13. A nucleic acid molecule encoding the chimeric antigen receptor of claim 1.

14. A vector comprising the nucleic acid molecule of claim 13.

15. A host cell, the host cell comprising the vector of claim 14.

16. A pharmaceutical composition comprising a pharmaceutically acceptable vector and the chimeric antigen receptor of claim 1.

17. (canceled)

18. (canceled)

19. A method for preparing a CAR-T cell, wherein the CAR-T cell expresses the chimeric antigen receptor of claim 1, and the method comprises the following step:
Introducing a nucleic acid molecule encoding the chimeric antigen receptor into a T cell so as to obtain the CAR-T cell.

20. A method of treating tumor in a subject, comprising administering to the subject a chimeric antigen receptor T cell (CAR-T cell) according to claim 9.

21. The method according to claim 20, wherein the tumor is a hematological tumor, preferably, the hematological tumor is B-cell malignancy, acute lymphocytic leukemia, chronic lymphocytic leukemia, lymphoma, mastocytoma or follicular lymphoma.

22. A method of treating tumor in a subject, comprising administering to the subject a nucleic acid molecule encoding a chimeric antigen receptor according to claim 13.


SEQUENCE LISTING (38 sequences)

SEQ ID NO: 1 - protein, 246 aa, Artificial Sequence
  Synthetic Construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 2 - DNA, 738 bp, Artificial Sequence
  Synthetic Construct
gacattgtgctgacccaatctccagctatcctgtctgcatctccaggggagaaggtcacaatgacttgcagggccagctcaagtgtaaattacatggactggtaccagaagaagccaggatcctcccccaaaccctggatttatgccacatccaacctggcttctggagtccctgctcgcttcagtggcagtgggtctgggacctcttactctctcacaatcagcagagtggaggctgaagatgctgccacttattactgccagcagtggagttttaatccacccacgttcggaggggggaccaagctggaaataaaaggcagtactagcggtggtggctccgggggcggttccggtgggggcggcagcagcgaggtgcagctgcagcagtctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagcctgacatctgaggactctgcggactattactgtgcaagatctaattattacggtagtagctactggttcttcgatgtctggggcgcagggaccacggtcacagtaagtagc

SEQ ID NO: 3 - protein, 246 aa, Artificial Sequence
  Synthetic Construct
EIVLTQSPATLSLSPGERATLSCRASSSVNYMDWYQQKPGQAPRLLIYATSNLASGIPARFSGSGSGTDFTLTISSLEPEDFAVYYCQQWSFNPPTFGQGTKLEIKGSTSGGGSGGGSGGGGSSQVQLVQSGAEVKKPGASVKVSCKASGYTFTSYNMHWVRQAPGQGLEWIGAIYPGNGDTSYNQKFKGRVTITRDTSASTAYMELSSLRSEDTAVYYCARSNYYGSSYWFFDVWGQGTLVTVSS

SEQ ID NO: 4 - DNA, 738 bp, Artificial Sequence
  Synthetic Construct
gagatcgtgctgacacagtctcccgccacactgtcactgtctccaggcgaaagagccacactgagctgtagagccagcagcagcgtgaactacatggactggtatcagcagaagcccggacaggcccctagactgctgatctacgccacaagcaatctggccagcggcatccctgccagattttctggctctggctccggcaccgatttcaccctgaccataagcagcctggaacctgaggacttcgccgtgtactactgccagcagtggtccttcaatcctcctacctttggccagggcaccaagctggaaatcaagggctctacaagcggcggaggatctggcggtggaagtggcggaggcggatcttctcaggttcagctggttcagtctggcgccgaagtgaagaaaccaggcgcctctgtgaaggtgtcctgcaaggcctctggctacacctttaccagctacaacatgcactgggtccgacaggctccaggacagggactcgaatggatcggcgccatctatcccggcaatggcgacacctcctacaaccagaaattcaagggccgcgtgaccatcaccagagacacatctgccagcaccgcctacatggaactgagcagcctgagaagcgaggataccgctgtgtactattgcgccagaagcaactactacggcagcagctactggttcttcgacgtgtggggacagggcaccctggtcacagtgtctagc

SEQ ID NO: 5 - protein, 245 aa, Artificial Sequence
  Synthetic Construct
DIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKLEIKGSTSGGGSGGGSGGGGSSQVTLKESGPALVKPTQTLTLTCTFSGFSLSDYGVSWIRQPPGKALEWLAVIWGSETTYYNSALKSRLTISKDTSKNQVVLTMTNMDPVDTATYYCAKHYYYGGSYAMDYWGQGTLVTVSS

SEQ ID NO: 6 - DNA, 735 bp, Artificial Sequence
  Synthetic Construct
gacatccagatgacccagagcccttcctccctgagcgcctccgtgggcgatagagtgacaatcacatgtagagcctcccaggacatcagcaagtacctgaactggtaccagcagaagcccggcaaggcccccaagctgctgatctaccacacctccagactgcacagcggcgtgcctagcaggttcagcggctccggcagcggcaccgactttacactgaccatcagctccctgcagcctgaggatttcgccacctactactgtcagcagggcaatacactgccctacacctttggccagggcaccaagctggagatcaagggatccaccagcggcggaggaagcggcggaggtagcggaggaggcggaagctcccaggtgacactgaaggagagcggccctgccctggtgaagcctacacagacactgacactgacgtgtaccttctccggcttcagcctgtccgattacggcgtgagctggatcagacagcctcctggcaaggccctggagtggctggccgtgatctggggcagcgagaccacctactacaattccgccctgaagagcaggctgaccatctccaaggacacctccaagaaccaggtggtgctgaccatgaccaatatggatcctgtggacaccgccacatactactgtgccaagcactactactacggcggcagctacgccatggattactggggccagggcaccctggtgaccgtgagctcc

SEQ ID NO: 7 - protein, 236 aa, Artificial Sequence
  Synthetic Construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 8 - DNA, 708 bp, Artificial Sequence
  Synthetic Construct
caggttcaactgcagcagtctggaccaggcctcgtgaagcctagccagacactgagcctgacatgtgccatcagcggcgatagcgtgtccagcaattctgccgcctggaactggatccggcagagcccttctagaggactcgagtggctgggcagaacctactatcggagcaagtggtacaacgactacgccgtgtccgtgaagtccaggatcaccatcaatcccgacaccagcaagaatcagttctccctgcagctgaacagcgtgacccctgaggataccgccgtgtactactgtgccagagaagtgaccggcgatctggaagatgccttcgacatctggggacagggcacaatggtcacagtgtcaagcggtggtggcggctccgatattcagatgacccagtctccttccagcctgtccgcctctgtgggcgatcgcgtgacaattacatgcagggccagccagaccatctggtcctatctcaattggtatcaacagcggcctggaaaggctcccaacctgcttatctatgccgcctccagtctgcagagcggagtgccttctagattcagcggaagaggaagcggcacagatttcacactgacaatcagctcactgcaggccgaagatttcgctacttactactgtcagcagagctacagcatccctcagacattcggccaggggacaaagctcgagattaag

SEQ ID NO: 9 - protein, 45 aa, Artificial Sequence
  Synthetic Construct
TTTPAPRPPTPAPTIASQPLSLRPEACRPAAGGAVHTRGLDFACD

SEQ ID NO: 10 - DNA, 135 bp, Artificial Sequence
  Synthetic Construct
accacaacacccgctcctagacctccaactcctgctccaaccattgccagtcaacccctgagtctgaggccagaggcatgcagaccagccgcaggcggagctgttcacactagaggcctggactttgcttgtgat

SEQ ID NO: 11 - protein, 24 aa, Artificial Sequence
  Synthetic Construct
IYIWAPLAGTCGVLLLSLVITLYC

SEQ ID NO: 12 - DNA, 72 bp, Artificial Sequence
  Synthetic Construct
atctatatttgggctccactggccgggacctgcggagttcttctgctgtctctcgtgatcacactctattgc

SEQ ID NO: 13 - protein, 42 aa, Artificial Sequence
  Synthetic Construct
KRGRKKLLYIFKQPFMRPVQTTQEEDGCSCRFPEEEEGGCEL

SEQ ID NO: 14 - DNA, 126 bp, Artificial Sequence
  Synthetic Construct
aagagaggccggaagaagctcctctatatctttaaacagccgtttatgcgcccggtccagacaacccaagaagaggacggctgtagctgtcggttccctgaagaggaagaaggcggttgcgaactg

SEQ ID NO: 15 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - DNA, 336 bp, Artificial Sequence
  Synthetic Construct
cgcgtgaaattctccagaagcgctgacgcccctgcttaccagcaaggccagaatcagctctataacgaactcaatctcggcaggcgcgaggaatatgatgtgctggataagaggcgcggcagggacccagagatgggaggaaagcctcggagaaagaacccacaagaaggactttacaacgaactgcaaaaggataagatggcagaagcttactccgagattggcatgaagggcgaacgtcggagaggaaaaggccacgacggactctatcagggactgtctacagccaccaaagacacctacgatgcactccatatgcaggctctgcctccacgg

SEQ ID NO: 17 - protein, 39 aa, Artificial Sequence
  Synthetic Construct
IEVMYPPPYLDNEKSNGTIIHVKGKHLCPSPLFPGPSKP

SEQ ID NO: 18 - DNA, 117 bp, Artificial Sequence
  Synthetic Construct
attgaagttatgtatcctcctccttacctagacaatgagaagagcaatggaaccattatccatgtgaaagggaaacacctttgtccaagtcccctatttcccggaccttctaagccc

SEQ ID NO: 19 - protein, 27 aa, Artificial Sequence
  Synthetic Construct
FWVLVVVGGVLACYSLLVTVAFIIFWV

SEQ ID NO: 20 - DNA, 81 bp, Artificial Sequence
  Synthetic Construct
ttctgggtgctggtggtggtgggaggagtgctggcctgctacagcctgctggtgaccgtggccttcatcatcttctgggtg

SEQ ID NO: 21 - protein, 41 aa, Artificial Sequence
  Synthetic Construct
RSKRSRLLHSDYMNMTPRRPGPTRKHYQPYAPPRDFAAYRS

SEQ ID NO: 22 - DNA, 123 bp, Artificial Sequence
  Synthetic Construct
aggagcaagaggagcaggctgctgcacagcgactacatgaacatgacccccagaaggcctggccccaccaggaagcactaccagccctacgccccccctagagacttcgccgcctacaggagc

SEQ ID NO: 23 - protein, 31 aa, Artificial Sequence
  Synthetic Construct
NLSIFDPPPFKVTLTGGYLHIYESQLCCQLK

SEQ ID NO: 24 - DNA, 93 bp, Artificial Sequence
  Synthetic Construct
aacctatcaatttttgatcctcctccttttaaagtaactcttacaggaggatatttgcatatttatgaatcacaactttgttgccagctgaag

SEQ ID NO: 25 - protein, 21 aa, Artificial Sequence
  Synthetic Construct
FWLPIGCAAFVVVCILGCILI

SEQ ID NO: 26 - DNA, 63 bp, Artificial Sequence
  Synthetic Construct
ttctggttacccataggatgtgcagcctttgttgtagtctgcattttgggatgcatacttatt

SEQ ID NO: 27 - protein, 38 aa, Artificial Sequence
  Synthetic Construct
CWLTKKKYSSSVHDPNGEYMFMRAVNTAKKSRLTDVTL

SEQ ID NO: 28 - DNA, 114 bp, Artificial Sequence
  Synthetic Construct
tgttggcttacaaaaaagaagtattcatccagtgtgcacgaccctaacggtgaatacatgttcatgagagcagtgaacacagccaaaaaatctagactcacagatgtgacccta

SEQ ID NO: 29 - protein, 36 aa, Artificial Sequence
  Synthetic Construct
RDQRLPPDAHKPPGGGSFRTPIQEEQADAHSTLAKI

SEQ ID NO: 30 - DNA, 108 bp, Artificial Sequence
  Synthetic Construct
cgagatcagcggctgcctcctgacgctcacaaacctccaggcggaggcagcttcagaacccctatccaagaggaacaggctgacgcccacagcacactggccaagatc

SEQ ID NO: 31 - protein, 229 aa, Artificial Sequence
  Synthetic Construct
ESKYGPPCPPCPAPPVAGGPSVFLFPPKPKDTLMISRTPEVTCVVVAVSQEDPEVQFNWYVDGVEVHNAKTKPREEQFASTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 32 - DNA, 687 bp, Artificial Sequence
  Synthetic Construct
gagtctaagtacggccctccttgtcctccatgtcctgctcctccagttgctggcggcccttccgtgtttctgttccctccaaagcctaaggacaccctgatgatcagcagaacccctgaagtgacctgcgtggtggtggccgtgtctcaagaggatcctgaggtgcagttcaattggtacgtggacggcgtggaagtgcacaacgccaagaccaagcctagagaggaacagttcgccagcacctacagagtggtgtccgtgctgaccgtggtgcatcaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttttacccctccgatatcgccgtggaatgggagagcaatggccagcctgagaacaactacaagaccacacctcctgtgctggacagcgacggctcattcttcctgtacagcaagctgacagtggacaagagccggtggcaagagggcaacgtgttctcctgtagcgtgatgcacgaggccctgcacaaccactacacccagaaaagcctgagcctgtctctgggcaag

SEQ ID NO: 33 - DNA, 885 bp, Artificial Sequence
  Synthetic Construct
gagtctaagtacggccctccttgtcctccatgtcctgctcctccagttgctggcggcccttccgtgtttctgttccctccaaagcctaaggacaccctgatgatcagcagaacccctgaagtgacctgcgtggtggtggccgtgtctcaagaggatcctgaggtgcagttcaattggtacgtggacggcgtggaagtgcacaacgccaagaccaagcctagagaggaacagttcgccagcacctacagagtggtgtccgtgctgaccgtggtgcatcaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttttacccctccgatatcgccgtggaatgggagagcaatggccagcctgagaacaactacaagaccacacctcctgtgctggacagcgacggctcattcttcctgtacagcaagctgacagtggacaagagccggtggcaagagggcaacgtgttctcctgtagcgtgatgcacgaggccctgcacaaccactacacccagaaaagcctgagcctgtctctgggcaagatctatatttgggctccactggccgggacctgcggagttcttctgctgtctctcgtgatcacactctattgcaagagaggccggaagaagctcctctatatctttaaacagccgtttatgcgcccggtccagacaacccaagaagaggacggctgtagctgtcggttccctgaagaggaagaaggcggttgcgaactg

SEQ ID NO: 34 - DNA, 891 bp, Artificial Sequence
  Synthetic Construct
gagtctaagtacggccctccttgtcctccatgtcctgctcctccagttgctggcggcccttccgtgtttctgttccctccaaagcctaaggacaccctgatgatcagcagaacccctgaagtgacctgcgtggtggtggccgtgtctcaagaggatcctgaggtgcagttcaattggtacgtggacggcgtggaagtgcacaacgccaagaccaagcctagagaggaacagttcgccagcacctacagagtggtgtccgtgctgaccgtggtgcatcaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttttacccctccgatatcgccgtggaatgggagagcaatggccagcctgagaacaactacaagaccacacctcctgtgctggacagcgacggctcattcttcctgtacagcaagctgacagtggacaagagccggtggcaagagggcaacgtgttctcctgtagcgtgatgcacgaggccctgcacaaccactacacccagaaaagcctgagcctgtctctgggcaagttctgggtgctggtggtggtgggaggagtgctggcctgctacagcctgctggtgaccgtggccttcatcatcttctgggtgaggagcaagaggagcaggctgctgcacagcgactacatgaacatgacccccagaaggcctggccccaccaggaagcactaccagccctacgccccccctagagacttcgccgcctacaggagc

SEQ ID NO: 35 - DNA, 864 bp, Artificial Sequence
  Synthetic Construct
gagtctaagtacggccctccttgtcctccatgtcctgctcctccagttgctggcggcccttccgtgtttctgttccctccaaagcctaaggacaccctgatgatcagcagaacccctgaagtgacctgcgtggtggtggccgtgtctcaagaggatcctgaggtgcagttcaattggtacgtggacggcgtggaagtgcacaacgccaagaccaagcctagagaggaacagttcgccagcacctacagagtggtgtccgtgctgaccgtggtgcatcaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttttacccctccgatatcgccgtggaatgggagagcaatggccagcctgagaacaactacaagaccacacctcctgtgctggacagcgacggctcattcttcctgtacagcaagctgacagtggacaagagccggtggcaagagggcaacgtgttctcctgtagcgtgatgcacgaggccctgcacaaccactacacccagaaaagcctgagcctgtctctgggcaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgacccta

SEQ ID NO: 36 - protein, 295 aa, Artificial Sequence
  Synthetic Construct
ESKYGPPCPPCPAPPVAGGPSVFLFPPKPKDTLMISRTPEVTCVVVAVSQEDPEVQFNWYVDGVEVHNAKTKPREEQFASTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGKIYIWAPLAGTCGVLLLSLVITLYCKRGRKKLLYIFKQPFMRPVQTTQEEDGCSCRFPEEEEGGCEL

SEQ ID NO: 37 - protein, 297 aa, Artificial Sequence
  Synthetic Construct
ESKYGPPCPPCPAPPVAGGPSVFLFPPKPKDTLMISRTPEVTCVVVAVSQEDPEVQFNWYVDGVEVHNAKTKPREEQFASTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGKFWVLVVVGGVLACYSLLVTVAFIIFWVRSKRSRLLHSDYMNMTPRRPGPTRKHYQPYAPPRDFAAYRS

SEQ ID NO: 38 - protein, 288 aa, Artificial Sequence
  Synthetic Construct
ESKYGPPCPPCPAPPVAGGPSVFLFPPKPKDTLMISRTPEVTCVVVAVSQEDPEVQFNWYVDGVEVHNAKTKPREEQFASTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGKFWLPIGCAAFVVVCILGCILICWLTKKKYSSSVHDPNGEYMFMRAVNTAKKSRLTDVTL